(19) United States
(12) Patent Application Publication
(10) Pub. No.: US 2026/0243745 A1
(43) Pub. Date: Aug. 20, 2026

(54) NON-LINEAR VALVE ADJUSTMENTS FOR LANDFILL GAS EXTRACTION

(71) Applicant: Loci Controls, Inc., Wareham, MA (US)

(72) Inventors: Ian Martin, Higganum, CT (US); Melinda Sims, Seattle, WA (US); Michael Gleick, Berkley, MA (US); Casey Campbell, Fairhaven, MA (US)

(21) Appl. No.: 19/536,720

(22) Filed: Feb. 11, 2026

Related U.S. Application Data
(60) Provisional application No. 63/758,476, filed on Feb. 14, 2025.

(51) Int. Cl. G01N 33/00; B09B 1/00

(52) U.S. Cl. CPC G01N 33/0047; B09B 1/006

(57) ABSTRACT

According to some aspects, there is provided a control system for controlling extraction of landfill gas from a landfill via a gas extraction system, the control system comprising: a valve configured to control flow rate of landfill gas extracted from the landfill; and at least one controller configured to: determine, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and open or close the valve by the determined degree.

### CROSS-REFERENCE TO RELATED APPLICATIONS

This application claims the benefit of priority under § 119(e) to U.S. Provisional Application Serial No. 63/758476 titled “NON-LINEAR VALVE ADJUSTMENTS FOR LANDFILL GAS EXTRACTION” and filed on Feb. 14, 2025, under Attorney Docket No. L0789.70023US00, which is incorporated by reference herein in its entirety.

### BACKGROUND

Landfills produce gas as a result of decomposition of organic waste in the landfill. The decomposition process may result in release of methane and other gases. Landfill sites are often capped with a layer of cover material to reduce the escape of gases from the landfill to the atmosphere. Landfills may further install gas extraction systems to pull landfill gas out before it can permeate through the cover layer and escape. The gas extraction systems may comprise multiple wells drilled into the landfill, and landfill gas may be extracted from the landfill via the wells into a gas collection system. The extracted landfill gas may be used to generate electricity, put in a pipeline for distribution, or disposed of.

### SUMMARY

According to some embodiments, there is provided a control system for controlling extraction of landfill gas from a landfill via a gas extraction system, the control system comprising: a valve configured to control flow rate of landfill gas extracted from the landfill; and at least one controller configured to: determine, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and open or close the valve by the determined degree.

According to some embodiments, there is provided a method for controlling extraction of landfill gas from a landfill via a gas extraction system comprising a valve configured to control flow rate of landfill gas extracted from the landfill, the method comprising: determining, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and opening or closing the valve by the determined degree.

According to some embodiments, there is provided at least one non-transitory computer-readable storage medium having encoded thereon instructions that, when executed by at least one processor, cause the at least one controller to perform a method for controlling extraction of landfill gas from a landfill via a gas extraction system comprising a valve configured to control flow rate of landfill gas extracted from the landfill, the method comprising: determining, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and opening or closing the valve by the determined degree.

### BRIEF DESCRIPTION OF DRAWINGS

Various aspects and embodiments will be described with reference to the following figures. It should be appreciated that the figures are not necessarily drawn to scale.

FIG. 1A shows an illustrative environment in which aspects of the technology described herein may be implemented.

FIG. 1B shows an example control system for landfill gas extraction, in accordance with some embodiments of the technology described herein.

FIG. 1C shows another example control system for landfill gas extraction, in accordance with some embodiments of the technology described herein.

FIG. 2A is a block diagram illustrating components of an example landfill gas extraction control system, in accordance with some embodiments of the technology described herein.

FIG. 2B is a block diagram of an example valve, in accordance with some embodiments of the technology described herein.

FIG. 2C is a schematic illustration of an illustrative embodiment of a valve having a movable member for restricting flow with the movable member in a state in which flow is enabled, in accordance with some embodiments of the technology described herein.

FIG. 3 is a graph depicting linearity of flow rate in response to changes in valve position.

FIG. 4 illustrates an example process for controlling extraction of landfill gas from a landfill using a technique that compensates for non-linearity in valve adjustments, in accordance with some embodiments of the technology described herein.

FIG. 5 is a graph depicting correction factors to be applied to valve adjustments, in accordance with some embodiments of the technology described herein.

FIG. 6 is a graph depicting ratios of flow rate changes to valve changes.

FIG. 7 is a graph comparing linearity of valves, according to some embodiments of the technology described herein.

FIG. 8 illustrates an example process for controlling extraction of landfill gas from a landfill using a technique that compensates for non-linearity in valve adjustments, in accordance with some embodiments of the technology described herein.

FIG. 9 is a graph depicting a linearization transfer function for use in determining a valve adjustment to achieve a target flow rate, according to some embodiments of the technology described herein.

FIG. 10 is a block diagram of an exemplary computer system in which aspects of the present disclosure may be implemented, according to some embodiments.

### DETAILED DESCRIPTION

Aspects of the technology described herein provide for systems and techniques which facilitate uniformly adjusting flow rate of landfill gas extracted from a landfill despite the non-linear nature of flow control valves. That is, the techniques provided herein compensate for the non-linearity in the relationship between the increment to which a valve is open or closed and the resulting change to flow rate of landfill gas through the valve.

Gas extraction systems control extraction of gas from a landfill to meet one or more objectives. For example, the objective(s) may include maximizing an energy content of gas extracted from the landfill (e.g., by maximizing an amount or concentration of methane in the gas, and/or by setting a flow rate to maximize the energy content), ensuring that extracted complies with regulations (e.g., government regulations), lowering an environmental impact of extracted gas, obtaining a specific composition of specific gases (e.g., methane, oxygen, carbon dioxide) in the extracted gas, and/or meeting an energy demand (e.g., of a power plant). Some gas extraction systems control flow of landfill gas from the landfill to a gas output to meet the objective(s). For example, gas extraction systems may control a flow rate of landfill gas extracted from the landfill to meet the objective(s).

Control of flow rate can be achieved by adjusting the degree to which a valve disposed in well piping of the gas extraction system is open or closed. For example, closing the valve to a greater degree decreases the flow rate of landfill gas extracted from the landfill while opening the valve to a greater degree increases the flow rate of landfill gas extracted form the landfill. The resulting change in flow rate impacts the characteristics (e.g., composition) of the landfill gas extracted from the landfill and the characteristics of the gas extraction system. For example, increasing the flow rate of landfill gas extracted from the landfill results in a decrease in methane concentration, and an increase in oxygen and nitrogen concentration of the landfill gas. By contrast, decreasing the flow rate of landfill gas extracted from the landfill results in an increase in methane concentration, and a decrease in oxygen and nitrogen concentration.

The inventors have recognized, however, that even high-quality valves typically have a disadvantage in that the relationship between the size of valve adjustments (e.g., in terms of a percentage of the valve's full range of motion) and the resulting change to flow rate is not linear along the range of the valve moving from fully opened to fully closed and vice versa. For example, due to the non-linearity, the resulting impact of a valve adjustment of a particular size on flow rate is different depending on the current position of the valve (e.g., the current degree to which the valve is open) when the adjustment is made. That is, a 5% increase to flow rate when the valve is at a nearly fully closed position results in a change in flow rate that is different from the change in flow rate resulting from a 5% increase to flow rate when the valve is between nearly fully closed and nearly fully opened (e.g., when the valve is approximately 50% open). Accordingly, a valve having a non-linear relationship between valve adjustments and flow rate changes is characterized in that the change in flow rate responsive to a valve adjustment depends not only on the degree to which the valve is opened or closed, but also on the current position of the valve. Conventional gas extraction systems do not account for such non-linearity. Rather, when it is determined to adjust flow rate, a valve is typically operated in standard increments (e.g., increments of 1%, 2%, 3%, 4%, 5%), regardless of the current position of the valve. Such existing systems do not account for the fact that uniform valve adjustments do not produce uniform changes in flow rate. Instead, multiple valve adjustments may need to be made in order to achieve the desired change in flow rate rendering the process of adjusting flow rate in conventional gas extraction systems inefficient and time intensive. Moreover, since conventional systems typically make valve adjustments according to a predefined schedule (e.g., on an hourly basis), it may take hours to implement the multiple valve adjustments and achieve the desired flow rate. This results in suboptimal extraction of landfill gas, reducing the amount of energy extracted from the well, increasing the risk of harmful environmental impacts such as greenhouse gas emissions and/or underground fires, or both.

The inventors have therefore developed techniques which compensate for non-linearity in the relationship between valve adjustments and flow rate changes. The techniques described herein provide for determination of corrected adjustments that achieve the desired change in flow rate, despite the non-linearity of the valve. Such techniques account for a current position of the valve and a desired change in flow rate when determining the appropriate valve adjustment to apply to the valve. The inventors have recognized that this non-linearity is exhibited by a wide variety of existing valves, including, for example, gate valves, ball valves including v-port ball valves, plug valves, and globe valves. The techniques described herein can be used to correct for non-linearity in any type of valve, including any of the valves described herein, regardless of how non-linear the relationship between valve adjustments and flow rate changes is for the particular valve. By compensating for the non-linearity of valve adjustments, the techniques described herein provide for more efficient gas extraction control. That is, the techniques described herein enable implementation of desired flow rate changes with fewer valve adjustments (e.g., in a single valve adjustment), and therefore in less time than conventional systems. As a result, landfill gas extraction is optimized, enabling greater amounts of energy to be extracted from the landfill, and the risk of dangerous conditions developing in the landfill (e.g., underground fires and/or greenhouse gas emissions) to be mitigated. In addition, the lifetime of the valve is extended as fewer mechanical movements (e.g., valve adjustments) are required to reach a desired flow rate, improving the longevity of the valve.

According to some embodiments, there is provided a control system for controlling extraction of landfill gas from a landfill via a gas extraction system, the control system comprising: a valve configured to control flow rate of landfill gas extracted from the landfill; and at least one controller configured to: determine, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and open or close the valve by the determined degree.

In some embodiments, the at least one controller is further configured to determine, based on the degree to which the valve is open at the time of the determining, a correction factor, and the determining the adjustment comprises applying the determined correction factor to the target change in flow rate to obtain the adjustment. In some embodiments, the determining the correction factor comprises using at least one lookup table that correlates valve positions with respective correction factors. In some embodiments, determining the correction factor further comprises selecting the at least one lookup table from a plurality of lookup tables based on a maximum flow rate of landfill gas being extracted from the landfill and/or a type of the valve.

In some embodiments, the at least one controller is further configured to determine the target change in flow rate of landfill gas through the valve. In some embodiments, the at least one controller is further configured to determine whether to adjust the flow rate of the landfill gas through the valve, wherein the determining the target change in flow rate through the valve is performed when it is determined to adjust the flow rate of the landfill gas through the valve. In some embodiments, the determining whether to adjust the flow rate of the landfill gas through the valve comprises determining, based on at least one characteristic of the landfill gas, whether to adjust the flow rate of the landfill gas through the valve based on at least one characteristic of the landfill gas. In some embodiments, the at least one characteristic of the landfill gas comprises a concentration of at least one constituent gas in the landfill gas and/or a pressure in the gas extraction system.

In some embodiments, the degree to which the valve is open at the time of the determining comprises a position of a throttle of the valve along a range from 0% to 100% open at the time of the determining. In some embodiments, the at least one controller is further configured to determine the position of the throttle at the time of the determining. In some embodiments, determining the position of the throttle at the time of the determining is performed based on a record of prior adjustments made to the valve.

In some embodiments, the at least one controller comprises: at least one first controller configured to determine the adjustment to apply to the valve; and at least one second controller configured to open or close the valve by the determined degree, wherein the at least one second controller is located remotely from the at least one first controller.

According to some embodiments, there is provided a method for controlling extraction of landfill gas from a landfill via a gas extraction system comprising a valve configured to control flow rate of landfill gas extracted from the landfill, the method comprising: determining, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and opening or closing the valve by the determined degree.

In some embodiments, determining, based on the degree to which the valve is open at the time of the determining, a correction factor, wherein the determining the adjustment comprises applying the determined correction factor to the target change in flow rate to obtain the adjustment. In some embodiments, the determining the correction factor comprises using at least one lookup table that correlates valve positions with respective correction factors. In some embodiments, the determining the correction factor further comprises selecting the at least one lookup table from a plurality of lookup tables based on a maximum flow rate of landfill gas being extracted from the landfill and/or a type of the valve.

In some embodiments, the method further comprises determining the target change in flow rate of landfill gas through the valve. In some embodiments, the method further comprises determining to adjust the flow rate of the landfill gas through the valve, wherein the determining the target change in flow rate through the valve is performed when it is determined to adjust the flow rate of the landfill gas through the valve. In some embodiments, the determining whether to adjust the flow rate of the landfill gas through the valve comprises determining, based on at least one characteristic of the landfill gas, whether to adjust the flow rate of the landfill gas through the valve based on at least one characteristic of the landfill gas. In some embodiments, the at least one characteristic of the landfill gas comprises a concentration of at least one constituent gas in the landfill gas and/or a pressure in the gas extraction system.

In some embodiments, the degree to which the valve is open at the time of the determining comprises a position of a throttle of the valve along a range from 0% to 100% open at the time of the determining. In some embodiments, the method further comprises determining the position of the throttle at the time of the determining. In some embodiments, the determining the position of the throttle at the time of the determining is performed based on a record of prior adjustments made to the valve.

According to some embodiments, there is provided at least one non-transitory computer-readable storage medium having encoded thereon instructions that, when executed by at least one processor, cause the at least one controller to perform a method for controlling extraction of landfill gas from a landfill via a gas extraction system comprising a valve configured to control flow rate of landfill gas extracted from the landfill, the method comprising: determining, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and opening or closing the valve by the determined degree.

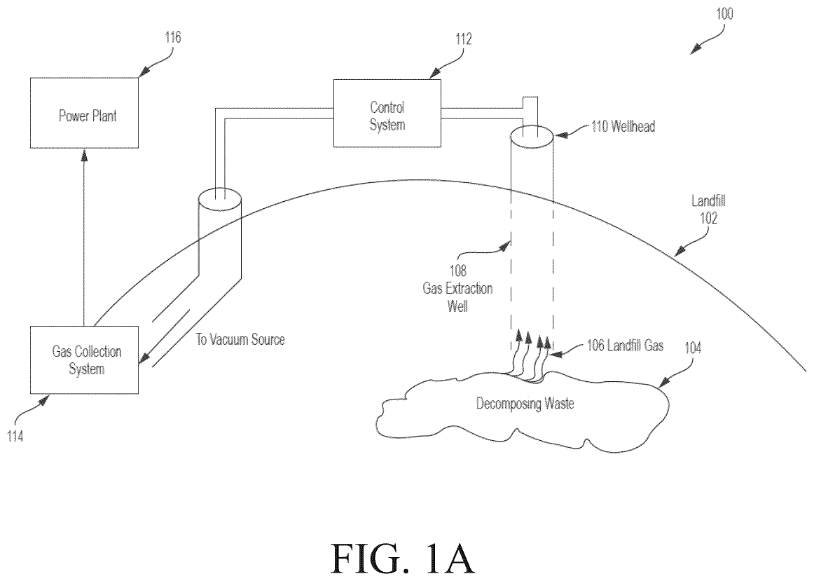

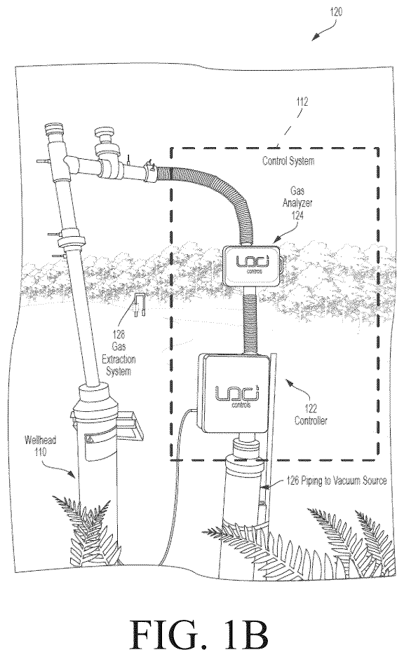

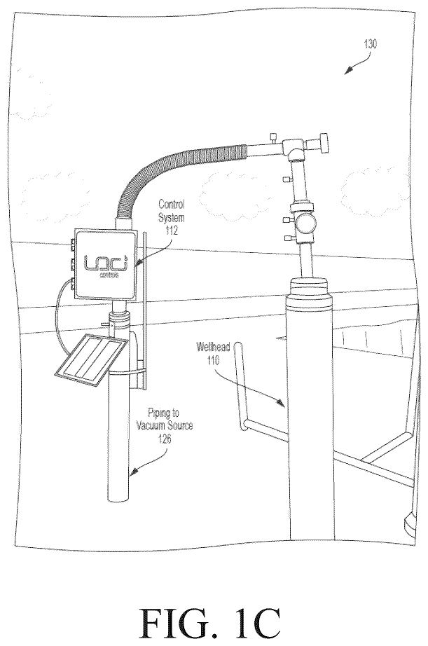

The aspects and embodiments described above, as well as additional aspects and embodiments, are described further below. These aspects and/or embodiments may be used individually, all together, or in any combination, as the application is not limited in this respect. FIG. 1A illustrates an example environment 100 in which aspects of the technology described herein may be implemented. The illustrative environment 100 includes a landfill 102 which holds decomposing waste 104. The decomposing waste 104 produces landfill gas (LFG) 106 which is extracted through a gas extraction well 108. The gas extraction well includes a wellhead 110 through which a control system 112 is coupled to the gas extraction well 108. The control system 112 may be configured to control extraction of gas via the gas extraction well 108. An output of the gas extraction system may be coupled to a gas collection system 114, which collects the landfill gas 106 extracted through the gas extraction well 108. The gas collection system 114 supplies the extracted landfill gas to a power plant 116. Although in the example embodiment shown in FIG. 1A, a single wellhead 110 is shown, in some embodiments, the environment 100 may include multiple wellheads at multiple sites. In such embodiments, the landfill gas may be extracted from the multiple sites.

In some embodiments, the gas collection system 114 includes a vacuum source. The vacuum source generates a negative pressure differential between the gas collection system 114 and the landfill 102. The negative pressure differential causes the landfill gas 106 to flow from the landfill 102 to the gas collection system 114 through the gas extraction well 108. In some embodiments, the gas collection system 114 may comprise an additional location where extracted landfill gas is stored, and where the extracted landfill gas may be treated (e.g., by removing impurities) before being supplied to the power plant or to the pipeline infrastructure 116. The power plant 116 may be configured to convert the extracted landfill gas into electrical power. For example, the power plant 116 may be configured to burn the extracted landfill gas to turn a rotor of an electricity generator or a turbine.

It should be appreciated, that although FIG. 1A illustrates supplying of extracted landfill gas from the collection system 114 to a power plant 116, the extracted landfill gas may additionally or alternatively be supplied to one or more other locations, and/or used for other purposes. For example, the gas collection system 114 may be configured to supply gas to existing gas pipelines, boilers, greenhouses, heating units, and/or other locations, as aspects of the technology described herein are not limited with respect to where the extracted landfill gas is supplied.

In some embodiments, the control system 112 controls extraction of the landfill gas 106 through the gas extraction well 108. In some embodiments, the control system 112 may be configured to operate to control extraction of landfill gas to achieve a desired outcome or outcomes with respect to energy content of extracted landfill gas, composition of extracted landfill gas, flow rate of gas extraction, regulatory requirements, and/or other parameters. In some embodiments, the control system 112 may include multiple components that operate to achieve the outcome(s), as discussed in more detail herein.

FIG. 1B illustrates an example implementation of the control system 112 for a landfill gas extraction system 120. The gas extraction well 108 may be coupled to the vacuum source through the piping 126 that leads to the vacuum source. Landfill gas may flow from the gas extraction well 108 towards the vacuum source via the piping 126. In some embodiments, the control system 112 is disposed within the piping 126 such that the control system 112 controls the flow of gas from the wellhead 110 to the vacuum source via the piping 126. The control system 112 includes a gas analyzer 124 which the control system 112 uses to determine one or more characteristics of the extracted landfill gas. The control system 112 includes a controller 122 that uses the determined characteristic(s) to control extraction of landfill gas. In some embodiments, the controller 122 may be configured to use the measured characteristic(s) to control a flow rate of landfill gas extraction. For example, the controller 122 may be configured to use the measured characteristic(s) to control a position of a valve that controls the flow rate of landfill gas being extracted.

In some embodiments, the gas analyzer 124 may be configured to collect and analyze extracted landfill gas. The gas analyzer 124 may be configured to include one or more sensors to measure the characteristic(s) of the extracted landfill gas. In some embodiments, the gas analyzer 124 may be configured to use the sensor(s) to measure composition, temperature, and/or other characteristic of the extracted landfill gas. In some embodiments, the gas analyzer may be configured to use the sensor(s) to measure the characteristic(s) of landfill gas when the gas is extracted (e.g., before being analyzed by the gas analyzer 124). For example, the sensor(s) may be used to measure concentration of methane, carbon dioxide, oxygen, and/or hydrogen sulfide. The sensor(s) may comprise, for example, infrared sensors, catalytic beads, electrochemical sensors, photoionization detectors, zirconium oxide sensors, thermal conductive detectors, and/or any other suitable sensing technology for measuring the characteristic(s) of the landfill gas, as aspects of the technology described herein are not limited to using a particular type of sensor.

In some embodiments, the gas analyzer 124 may be configured to determine one or more characteristics of the environment (e.g., ambient temperature, atmospheric pressure, wind direction, wind speed, precipitation, humidity), and/or gas in the landfill (e.g., temperature, composition, humidity). The gas analyzer 124 may include one or more sensors to obtain measurements of the characteristic(s). The sensors can include, for example, temperature sensors, humidity sensors, pH sensors, pressure sensors and/or any other type of sensor(s) for sensing environmental characteristics.

In some embodiments, the controller 122 may be configured to control one or more parameters of landfill gas extraction. In some embodiments, the controller 122 may be configured to control a flow rate of landfill gas being extracted from the landfill 102. In some embodiments, the control system 112 may include a flow control mechanism (e.g., a valve comprising a throttle) to control a flow rate of landfill gas extraction. For example, the control system 112 may include a valve that includes an actuation mechanism for changing the position of a throttle of the valve to control the flow rate. The controller 122 may be configured to determine and apply settings to the valve to control the flow rate of landfill gas extraction (e.g., operate the actuation mechanism to change the position of the throttle to a determined position). In some embodiments, the control mechanism is placed between the gas extraction well 108 and the gas collection system 114 such that gas being extracted through the gas extraction well 108 flows through the control mechanism on its way to the gas collection system 114.

In some embodiments, the throttle may be configured to block a variable portion of an aperture through which landfill gas extracted from the landfill flows. The position of the throttle may determine the variable portion of the aperture through which the landfill gas flows. In some embodiments, the throttle may have a position of maximum closure at which it allows at least some flow. In some embodiments, at the position of maximum closure the throttle may be configured to block a maximum portion of the aperture that is less than a full area of the aperture to allow at least some flow of landfill gas through the aperture when the throttle blocks the maximum portion. In some embodiments, the throttle may include a separate channel for at least some landfill gas to flow at the position of maximum closure.

In some embodiments, the maximum variable portion of the aperture that can be blocked is less than or equal to a percentage of the area of the aperture. In some embodiments, the maximum variable portion is less than 100% of the area of the aperture. In some embodiments, the maximum variable portion is less than 99%, 98%, 97%, 96%, or 95% of the area of the aperture. In some embodiments, the maximum portion of the aperture occluded when the throttle is in a position of maximum closure may be between 90% and 99% or between 85% to 99% or between 85% and 95%, in various embodiments. By limiting the variable portion to be less than the area of the aperture, the throttle may prevent the flow of landfill gas from completely shutting off, and thus prevent the gas output from going to a positive pressure relative to the landfill.

In some embodiments, the component that is moved to block the aperture may have an area that is greater than or equal to 100% of the area of the aperture. In these embodiments, the throttle may not shut off the flow of landfill even at a position of maximum closure. The throttle may not completely seal off flow, and thus will still allow at least some flow of landfill gas through the aperture, and thus prevent the gas output from going to a positive pressure.

In some embodiments, the throttle may be configured to allow at least a portion of the maximum flow rate. The maximum flow rate may comprise a flow rate of landfill gas through an aperture without any portion of the aperture being blocked. In some embodiments, the throttle may be configured to allow at least 1% of the maximum flow rate, regardless of operating state of the throttle. In some embodiments, the throttle may be configured to allow at least 2%, 3%, 4%, or 5% of the maximum flow rate. In some embodiments, the throttle may be configured to allow less than 20% of the maximum flow rate at a position of maximum closure. In some embodiments, the throttle may be configured to allow less than 10% of the maximum flow rate at the position of maximum closure. In some embodiments, the throttle may be configured to allow less than 5% of the maximum flow rate at the position of maximum closure.

In some embodiments, the throttle may be configured to allow at least a minimum flow rate of the landfill gas. In some embodiments, the throttle may be configured to allow at least 0.1 cubic feet per minute. In some embodiments, the throttle may be configured to allow at least 0.2, 0.3, 0.4, 0.5, 0.6, 0.7, 0.8, 0.9, or 1 cubic feet per minute of landfill gas flow. In some embodiments, the throttle may be configured to allow a minimum flow rate of any value between 1 and 5 cubic feet per minute. In some embodiments, the throttle may be configured to allow 0.1-120 cubic feet per minute, 0.1-150 cubic feet per minute, 0.1-175 cubic feet per minute, or 0.1-200 cubic feet per minute. Herein, cubic feet per minute are standard cubic feet per minute.

In some embodiments, the throttle may include a motor that generates a force by which to adjust the position of the throttle. The controller 122 may be configured to control the position of the throttle using the motor. In some embodiments, the controller 122 may be configured to control a flow rate of landfill gas extracted from the landfill by controlling the position of the throttle. For example, by changing the throttle position to increase the portion of the aperture that is blocked, the controller 122 may increase the flow rate of landfill gas; and by changing the plate position to decrease the portion of the aperture that is blocked by the plate, the controller 122 may decrease the flow rate of landfill gas.

In some embodiments, the controller 122 may be coupled to the gas analyzer 124. The controller 122 may be configured to use measurements obtained by the gas analyzer 124 to determine the control parameter(s). In some embodiments, the controller 122 may be configured to regulate the landfill gas flow rate based on the measurements obtained by the gas analyzer 124. To adjust the flow rate, in some embodiments, the controller 122 may be configured to adjust a throttle position to modify the flow rate. The controller 122 may be configured to control an actuation mechanism (e.g., a motor) to move the position of the throttle in order to obtain a desired position of the throttle to achieve a particular flow rate and/or cause a particular change in flow rate. In some embodiments, the controller 122 may be configured to determine a target flow rate based on the measurements of the characteristic(s) obtained by the gas analyzer 124. The controller 122 may be configured to adjust the throttle such that the flow rate is the target flow rate.

Example systems and techniques for controlling extraction of landfill gas are described in U.S. Patent Application Publication No. 2017/0216893, entitled “DEVICES AND TECHNIQUES RELATING TO LANDFILL GAS EXTRACTION” filed on Mar. 13, 2017, U.S. Patent Application Publication No. 2022/0008970, entitled “DEVICES AND TECHNIQUES RELATING TO LANDFILL GAS EXTRACTION” filed on Jul. 13, 2020, each of which are incorporated herein by reference. Some embodiments may include one or more features of embodiments described in the referenced applications.

In some embodiments, multiple wells or gas extraction systems may be located at a landfill to extract gas from the landfill. For example, FIG. 1B illustrates another well and gas extraction system 128 located at the landfill. In some embodiments, multiple gas extraction systems at the landfill may include the control system 112 for controlling extraction of landfill gas from the landfill. For example, gas extraction system may include the control system 112 to control extraction of landfill gas via the gas extraction system 128.

Although the gas analyzer 124 and the controller 122 are shown as separate components in FIG. 1A, in some embodiments, the gas analyzer 124 and controller 122 may be portions of a single unit. Some embodiments are not limited to any particular arrangement or combination of the gas analyzer 124 and the controller 122. In some embodiments, functionality described for each of the gas analyzer 124 and the controller 122 may be interchanged between the two components, as some embodiments of the technology described herein are not limited in this respect.

FIG. 1C illustrates an example implementation of the control system 112 for a landfill gas extraction system 130. In some embodiments, the gas analyzer and the controller described with reference to FIG. 1B are portions of the control system 112 shown in FIG. 1C. The gas extraction well 108 may be coupled to the vacuum source through the piping 126 that leads to the vacuum source. Landfill gas may flow from the gas extraction well 108 towards the vacuum source via the piping 126. In some embodiments, the control system 112 is disposed within the piping 126 such that the control system 112 controls the flow of gas from the wellhead 110 to the vacuum source via the piping 126. In some embodiments, the control system 112 may be configured to operate as described above with reference to FIG. 1B. For example, the control system 112 may be configured to use a gas analyzer and controller in the control system 112 to obtain measurements of one or more characteristics of the landfill gas being extracted via the gas extraction system and control extraction of the gas based on the measurements of the characteristic(s).

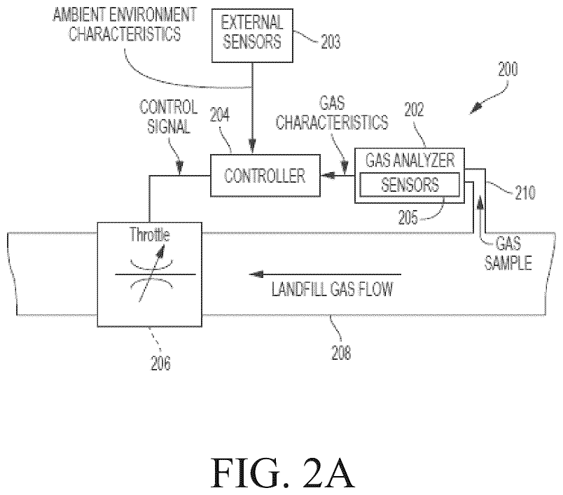

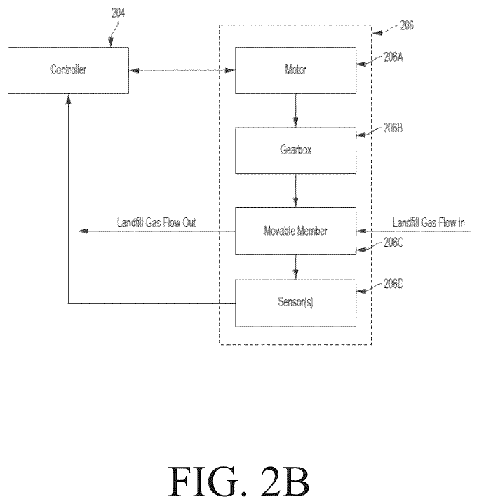

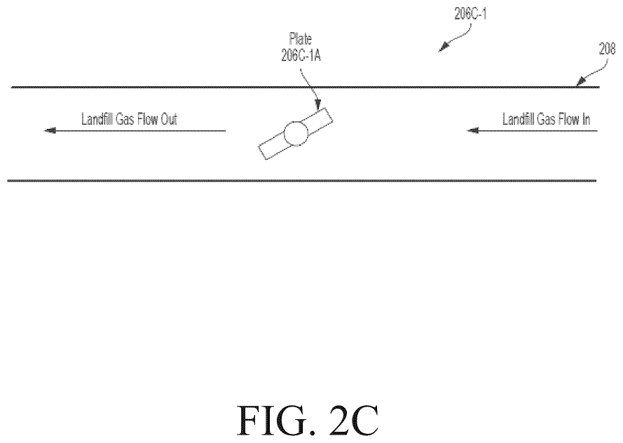

FIG. 2A illustrates a block diagram of components of an example control system 200 for controlling extraction of gas via a gas extraction system, according to some embodiments of the technology described herein. In some embodiments, control system 200 may be a portion or all of control system 112 discussed above with respect to FIGS. 1A-1C. For example, in some embodiments, the control system 200 may be configured to control flow of landfill gas from the landfill 102 through gas extraction well 108.

In some embodiments, the control system 200 includes a gas analyzer 202 for measuring one or more characteristics of landfill gas being extracted from the landfill. Measurements of the characteristic(s) of the landfill gas being extracted from the landfill may be used by a controller 204 to control a valve 206. In some embodiments, the valve 206 may be configured to control flow of landfill gas from the landfill to a gas collection system (e.g., gas collection system 114). In some embodiments, the valve 206 may be configured to control an aperture through which landfill gas from the landfill flows when flowing towards the gas collection system. For example, the valve 206 may be configured to control a variable portion of the aperture that is blocked in order to control a flow rate of the landfill gas flowing from the landfill to the gas collection system.

In some embodiments, the gas analyzer 202 may be configured to determine one or more characteristics of landfill gas and/or a surrounding environment of the landfill. For example, the gas analyzer 202 may be configured to determine the characteristic(s) of extracted landfill gas, gas in the landfill, landfill gas in different portions of the gas extraction system, and/or landfill gas in a gas collection system. In some embodiments, the gas analyzer 202 includes one or more sensors 205 to obtain measurements of the characteristic(s). In some embodiments, the gas analyzer 202 may be configured to obtain a sample of landfill gas from the gas extraction system 208 via an input port 210. The gas analyzer 202 may be configured to obtain measurements of the characteristic(s) of the collected gas sample using the sensor(s) 205. In some embodiments, the gas analyzer 202 may be configured to report the measurements of the characteristic(s) to the controller 204 for use in controlling the flow control mechanism 206.

In some embodiments, the gas analyzer 202 may be configured to obtain measurements for one or more characteristics of a collected gas sample or of landfill gas in the landfill. For example, the gas analyzer 202 may be configured to determine a temperature, pressure, flow rate, humidity, density, gas composition (e.g., concentration of methane, oxygen, carbon dioxide, carbon monoxide, hydrogen sulfide, nitrogen, and/or other gas) and/or any other suitable characteristic(s) of the collected gas sample. In some embodiments, the gas analyzer 202 may be configured to determine one or more characteristics of the landfill gas based on measurements obtained for a number of gas samples. For example, the gas analyzer 202 may be configured to determine an energy content of gas, or a concentration of methane in gas samples for a certain time period and/or across a number of collected gas samples. In some embodiments, the gas analyzer 202 may be configured to determine the characteristic(s) at a regular frequency. For example, the gas analyzer 202 may be configured to determine the characteristic(s) every second, every minute, every hour, every 12 hours, every 24 hours, every week, or at another frequency.

In some embodiments, the gas analyzer 202 may be configured to determine a gas composition of landfill gas being extracted from the landfill. In some embodiments, the gas analyzer 202 may be configured to determine a concentration of methane in a collected sample of gas. For example, the gas analyzer 202 may be configured to use the sensor(s) 205 to measure the concentration of methane in the sample of gas. The gas analyzer 202 may be configured to determine, using the determined concentration of methane, energy content of landfill gas being extracted from the landfill. The gas analyzer 202 may be configured to output the determined energy content to the controller 204 which may be configured to use the energy content to control the valve 206.

In some embodiments, the control system 200 may include one or more external sensors 203 to measure one or more characteristics of the ambient environment outside of Gas Analyzer 202 (e.g., outside of the control system 200). The external sensor(s) 203 may provide obtained measurements to the control system 200 (e.g., to controller 204) and/or to one or more computing devices located remotely (e.g., by using a wireless links, a wired link, and/or any suitable combination of wireless and wired links). In some embodiments, external sensor(s) 203 may include one or more temperature sensors configured to measure temperature outside of the control system 200 (e.g., the ambient atmospheric temperature) and/or any other suitable location. In some embodiments, the external sensor(s) 203 may include one or more atmospheric pressure sensor(s) configured to measure atmospheric pressure outside of the control system 200 (e.g., ambient atmospheric pressure) and/or any other suitable location. In some embodiments, the external sensor(s) 203 may include one or more humidity sensors to measure ambient humidity outside of the landfill.

In some embodiments, the control system 200 may include one or more sensors placed directly in gas extraction piping to obtain measurements of characteristics of the gas at different stages of the extraction system. In some embodiments, the control system 200 may include remote components (e.g., a computing device) for processing data to obtain the measurement(s) of the gas characteristic(s).

In some embodiments, the controller 204 may be configured to determine one or more settings of one or more control parameters and/or apply the control parameter(s) to the valve 206. In some embodiments, the controller 204 may be configured to use measurements of one or more gas characteristics (e.g., energy content of landfill gas, temperature of landfill gas) determined by the gas analyzer 202 to control the flow of landfill gas extracted from the landfill In some embodiments, the controller 204 may be configured to determine a flow rate at which landfill gas is to flow through the gas extraction system 208. The flow rate may comprise a target flow rate for extracting landfill gas. In some embodiments, the controller 204 may be configured to determine the target flow rate based on energy content of landfill gas being extracted from the landfill. If the energy content of the landfill gas being extracted is different from a target energy content, the controller 204 may be configured to adjust the flow rate to change the energy content of landfill gas being extracted from the landfill.

In some embodiments, the valve 206 may include a mechanism by which to block a portion of an aperture through which landfill gas extracted from the landfill flows. In some embodiments, the valve 206 may include a plate that blocks a variable portion of the aperture. The valve 206 may be configured to control a position of the plate to control the variable portion of the aperture that is blocked. The plate, however, may have an area that is less than the cross sectional area of an aperture in the well piping. Accordingly, even when in a position of maximum closure, the plate does not fully block the flow of gas. In some embodiments, the plate may have an area that is greater than or equal to the cross sectional area of the aperture in the well piping. The plate may not completely seal off flow in the position of maximum closure. The aperture, for example, may be fully or partially bounded by shoulders against which the plate may rest in the position of maximum closure. The plate and surface(s) of the shoulders, may be configured so as not to form an airtight seal. As a result, the plate allows at least some flow of gas through the aperture.

Other structures may be used to control flow without fully blocking the flow. In some embodiments, the valve 206 may include a ball valve that blocks a variable portion of the aperture. In some embodiments, the valve 206 may be configured to allow at least some flow of landfill gas through the aperture when in a state of maximum closure. Examples of mechanisms by which to block the portion of the aperture are discussed herein.

In some embodiments, the valve 206 may include one or more actuation devices configured to physically operate the valve 206. For example, in embodiments in which the throttle includes a plate positioned within an aperture of a pipe, the actuation device(s) may be configured to rotate the plate, thus altering the percentage of the opening of the pipe blocked by the plate. For example, the actuation device(s) may comprise a motor that uses electrical power to adjust the rotational position of the plate. In another example, the actuation device(s) may comprise a pneumatic actuator that uses air pressure to act on a piston to adjust the position of the plate. In yet another example, the actuation device(s) may comprise a hydraulic actuator that uses hydraulic pressure to adjust the position of the plate. Some embodiments are not limited to any specific type of actuation device, as any actuation device suitable for a respective valve 206 may be used.

In some embodiments, to change flow rate, the controller 204 may be configured to control a position of the valve 206. In some embodiments, the controller 204 may be configured to determine the throttle position to set based on a target flow rate of landfill gas flowing through the gas extraction system 208. For example, the target flow rate may correspond to a setting that may achieve a target energy content in landfill gas being extracted from the landfill. In some embodiments, the controller 204 may be configured to control one or more actuation devices to apply the parameter(s) to the valve 206. For example, in a system in which the actuation device(s) comprises a motor, the controller 204 may be configured to use the motor to change the throttle position.

Although, in the example embodiment illustrated in FIG. 2A, the controller 204 is shown to be co-located with the landfill gas extraction system 208, in some embodiments one or more components of the controller 204 may be remote from the physical gas extraction system 208. For example, the controller 204 may include a computing device configured to perform flow control calculations and communicate settings remotely to a device disposed in piping between a gas extraction well and a vacuum source. The device may be configured to control the flow control mechanism based on settings received from the computing device. The computer device may be configured to communicate settings via wireless communication or by wired communication. In another example, the computing device may be configured to remotely control one or more actuation devices to adjust positions of one or more valves of the flow control mechanism.

FIG. 2B illustrates components of the valve 206 shown in FIG. 2A according to some embodiments. The valve 206 includes a motor 206A, a gearbox 206B, a movable member 206C, and one or more sensors 206D. The controller 204 controls the motor 206A to modulate a position of the movable member 206C to control a flow rate of landfill gas flowing through the throttle (e.g., through the movable member). The gearbox 206B translates motion generated by the motor 206A to the movable member 206C causing the movable member 206C to change position. In some embodiments, the sensor(s) 206D measure a flow rate of landfill gas through the movable member 206C. In some embodiments, the sensor(s) 206D measure a pressure upstream of the valve 206, downstream of the valve 206, and/or a difference between the pressure upstream and downstream of the valve 206. The controller 204 uses one or more measurements of flow rate obtained by the sensor(s) 206D for controlling the motor 206A.

In some embodiments, the motor 206A provides a force by which the position of the throttle can be adjusted. The controller 204 may be configured to command the motor to adjust a position of the movable member 206C. For example, the motor 206A may generate a torque by which the movable member 206C may be moved to adjust the position of the movable member 206C. In some embodiments, the motor 206A may comprise a step motor that divides a full rotation of the motor into a number of equal steps. The controller 204 may be configured to command the motor to move and/or hold at one of the steps.

In some embodiments, the gear box 206B may be configured to translate a force generated by the motor 206A to the movable member 206C to adjust the position of the movable member 206C. The gear box 206B may be coupled to the movable member 206B to allow the force generated by the motor 206A to be translated to the movable member 206B and cause movement of the movable member 206B. In some embodiments, the gear box 206B is mechanically coupled to the movable member 206C. For example, the gear box may be attached to the movable member 206C via shaft. When the motor 206A generates a force, the force is translated to the shaft via the gear box 206B, which causes the shaft to move. The movement of the shaft then causes the movable member 206C to move. In some embodiments, the gear box 206B may be magnetically coupled to the movable member 206C.

In some embodiments, the movable member 206C may be configured to restrict a flow of landfill gas. In some embodiments, the movable member 206C may block a variable portion of an aperture through which the landfill gas flows. A position of the movable member may set the variable portion of the aperture. In some embodiments, the controller 204 may control the position of the movable member to set a flow rate of the landfill gas extracted from a landfill gas extraction system. For example, the landfill gas may flow through the movable member, and the controller 204 may control a position of the movable member to set the variable portion of the aperture, which in turn sets the flow rate of the landfill gas flowing through the movable member. The movable member may have a position of maximum closure in which the movable member provides a maximum amount of restriction to the flow of landfill gas. For example, the position of maximum closure may be a position at which the movable member blocks a maximum portion of the aperture through which the landfill gas flows. In some embodiments, the movable member may be configured to ensure that at least some flow is allowed through the movable member at the position of maximum closure.

FIG. 2C illustrates one example of a movable member 206C-1 in accordance with some embodiments. Although FIG. 2C illustrates one example of a movable member 206C-1, it should be appreciated that other types of valves may be implemented with the techniques described herein.

In the illustrated embodiment of FIG. 2C, the movable member 206C-1 may be the movable member 206C in the valve 206 of FIGS. 2A-B. The movable member 206C-1 may include a plate 206C-1A that is configured to rotate. The rotational position of the plate 206C-1A may set a flow rate of landfill gas extracted via the gas extraction system 208. In some embodiments, the controller 204 can be configured to command the motor 206A to generate a force that actuates the plate 206C-1A and causes the plate 206C-1A to rotate. In some embodiments, the rotational position of the plate 206C-1A may control a variable portion of an aperture through which the landfill gas flows. The plate 206C-1A may have a position of minimum restriction, and a position of maximum restriction. In some embodiments, the position of minimum restriction may correspond to a maximum flow rate that can be set by the flow control mechanism 206C-1, while the position of maximum restriction may correspond to a minimum flow rate that can be set by the flow control mechanism 206C-1.

As described herein, for many of the valves used in landfill environments, the relationship between valve adjustments (e.g., changes to the degree to which the valve is open or closed) and flow rate changes is non-linear. For example, a valve that has a linear relationship between valve adjustments and flow rate changes would produce the same change in flow rate responsive to a valve adjustment regardless of the current position of the valve. That is, if the valve were opened by 5%, the resulting change in flow rate would be the same regardless of whether the current position of the valve is nearly fully closed (e.g., 90% closed), nearly fully opened (e.g., 10% closed, or anywhere in between (e.g., 50% closed). By contrast, a valve having a non-linear relationship between valve adjustments and flow rate changes is characterized in that the change in flow rate responsive to a valve adjustment depends not only on the degree to which the valve is opened or closed, but also on the current position of the valve.

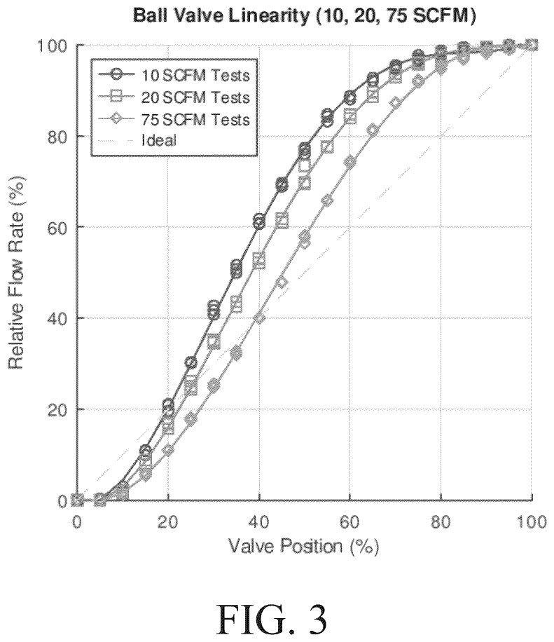

The relationship between valve adjustments and flow rate changes is illustrated in FIG. 3. In particular, FIG. 3 is a graph depicting linearity of flow rate in response to changes in valve position for a valve under application of three different maximum flow rates (10 SCFM, 20 SCFM, and 75 SCFM). To test the valve under the three different maximum flow rates, the valve was initially set to fully open, and the vacuum blower of the landfill gas extraction system was adjusted until the landfill gas flowing through the valve was at or near the desired flow rate, referred to herein as the maximum flow rate. In this case, the desired flow rates were selected to represent conditions of low flow at 10 SCFM, medium flow at 20 SCFM, and high flow at 75 SCFM. Then, the valve was closed in 5% increments until fully closed. Accordingly, the 10 SCFM, 20 SCFM, and 75 SCFM conditions represent maximum flow rates at which landfill gas can be extracted from the landfill under the present vacuum blower settings when the valve is fully open. In each case, the relationship between valve adjustments and flow rate change is non-linear. That is, the relative change in flow rate responsive to a change in valve position is represented in FIG. 3 by the slope of the curve at a particular valve position. As can be seen in FIG. 3, the slopes of the lines differ along the range of valve opening from 0% to 100%. For example, an adjustment to the valve produces relatively little change in flow rate when the current position of the valve is between 0-20% open or 80-100% open. By contrast, an adjustment to the valve when the current valve position is between 20-80% open produces a greater change in flow rate.

FIG. 3 further illustrates an ideal valve having a linear relationship between valve adjustments and flow rate changes regardless of the current position of the valve. In such an ideal case, valve adjustments are equally effective to implement a change in flow rate along the entire range of the valve positions from 0-100% open. The ideal case where the valve has the linear relationship between valve adjustments and flow rate changes avoids the inefficiency of valve adjustments producing relatively less change in flow rate at the ends of the range of valve positions.

The inventors have recognized that one way to address this non-linear valve behavior is to compensate for it by determining corrected adjustments based on both a desired flow rate change and the initial position of the valve at the time the adjustment is being made (whereas conventional methods for valve adjustment in this context do not take the valve position into account). The resulting techniques facilitate making uniform changes to flow rate despite that non-linearity of the valve.

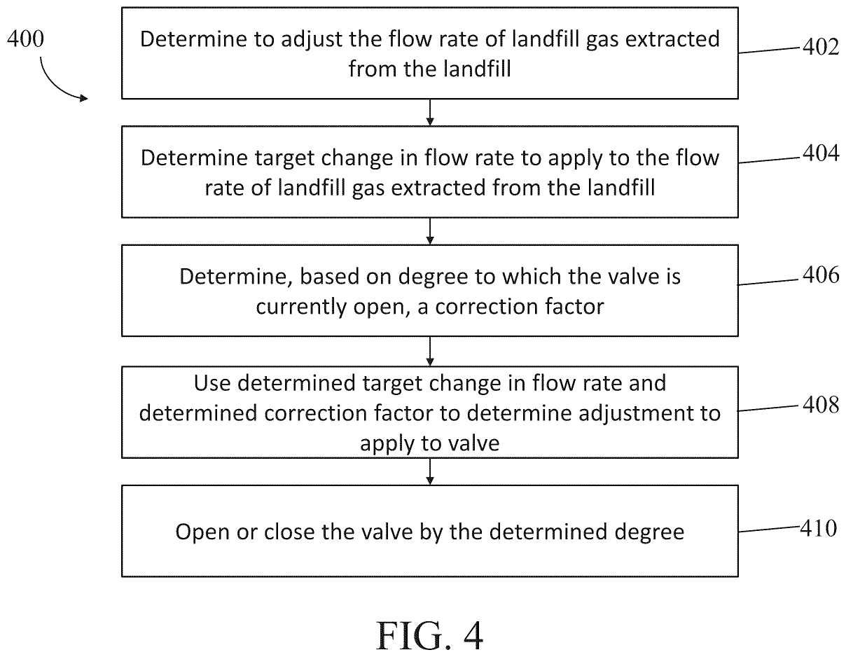

FIG. 4 illustrates an example process 400 for controlling extraction of landfill gas from a landfill using a technique that compensates for non-linearity in valve adjustments, in accordance with some embodiments of the technology described herein. The example process begins at act 402, where it is determined to adjust the flow rate of landfill gas extracted from the landfill. The determination may include determining whether to increase or decrease flow rate. The determination to adjust the flow rate of landfill gas extracted from the landfill may be performed in any suitable way. For example, in some embodiments, the determination to adjust the flow rate may be based on a characteristic of the gas extraction system and/or the extracted landfill gas, examples of which are provided herein. For example, in some embodiments, the determination to adjust the flow rate may be based on a concentration of one or more gasses in the extracted landfill gas (e.g., methane, oxygen, nitrogen, balance gas, carbon dioxide, hydrogen sulfide, a calculation of energy content) and/or based on a pressure in the gas extraction system.

When it is determined to adjust the flow rate of landfill gas extracted from the landfill, the process 400 moves to act 404, where a target change in flow rate to apply to the flow rate of landfill gas extracted from the landfill is determined. For example, in some embodiments, the target change to flow rate may be a standard incremental adjustment. That is, when it is determined to adjust the flow rate of landfill gas extracted from the landfill, the target change in flow rate may be adjusted by this amount. The amount may be predefined or dynamically defined during performance of the process 400, for example, based on one or more characteristics. In some embodiments, the predefined amount may be a percentage change by which to increase or decrease flow rate (e.g., 1%, 2%, 3%, 4%, 5%, 6%, 7%, 8%, 9%, 10%, etc.). In some embodiments, the predefined amount may be a fixed amount by which to increase or decrease flow rate. In some embodiments, the target change to flow rate may not be predefined, but rather may be dynamically determined based on one or more factors, such as a characteristic of the well from which landfill gas is extracted (e.g., an amount of cover at the well), a characteristic of the environment of the landfill (e.g., a measure of atmospheric pressure, temperature, humidity, wind speed, a gas concentration in the atmosphere, or other suitable characteristic), a characteristic of the gas extraction system (e.g., a measure of pressure in the gas extraction system), and/or a characteristic of the greenhouse gas extracted from the landfill (e.g., a concentration such as methane, nitrogen, balance gas, oxygen, carbon dioxide, hydrogen sulfide; an energy content, or other suitable characteristic). As an example, the target change to flow rate may be based on the difference between a target value for a characteristic and a current value for the characteristic.

The method then moves to act 406, where a correction factor is determined. As described herein, the correction factor is used in act 408 to determine the adjustment to apply to the valve. The correction factor may be a coefficient that is applied to a valve adjustment to obtain a corrected valve adjustment to apply to the valve. In some embodiments, the correction factor may be less than one and the corrected valve adjustment is lesser in magnitude than the initial uncorrected valve adjustment. In some embodiments, the correction factor may be greater than one and the corrected valve adjustment is greater in magnitude than the initial uncorrected valve adjustment. In some embodiments, the correction factor is equal to one, and the corrected valve adjustment is equal to the initial uncorrected valve adjustment.

In some embodiments, the correction factor may be determined based on a degree to which the valve is currently open. As described herein, for valves having a non-linear relationship between valve adjustments and changes to flow rate, the change in flow rate responsive to a valve adjustment depends not only on the valve adjustment, but also on the current degree to which the valve is open (or, equivalently, closed). The degree to which the valve is open may be represented as a percent to which the valve is open within a range from 0-100% open. In some embodiments, the degree to which the valve is open comprises a current position of a throttle of the valve along a range from 0% to 100% open.

In some embodiments, the method may include an act of determining the degree to which the valve is open. For example, the determining the degree to which the valve is open may be based on a record of prior adjustments made to the valve. By tracing the prior adjustments made to the valve (e.g., in percentages), the current degree to which the valve is open may be determined. Accordingly, in some embodiments, the method further includes an act of storing a record of prior adjustments made to the valve and the determining the degree to which the valve is open is performed based on the record of prior adjustments made to the valve.

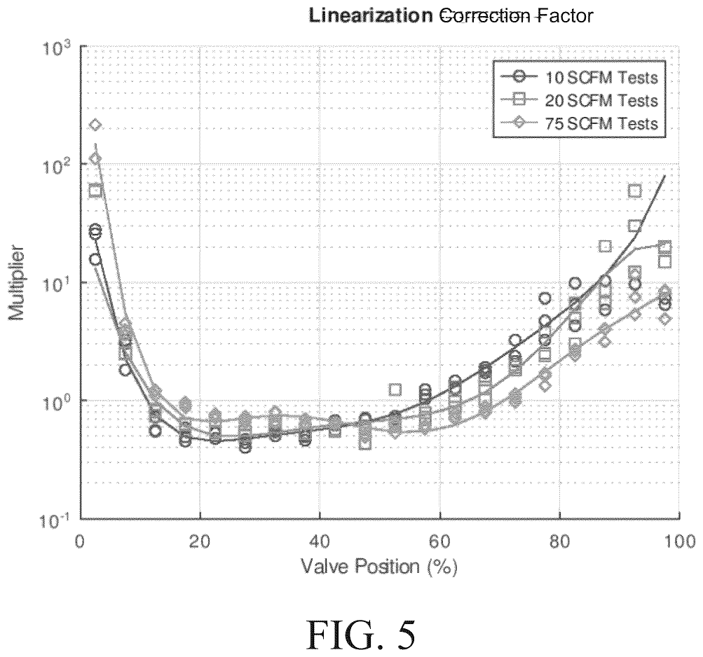

As described herein, the degree to which the valve is open is used to determine the correction factor. For example, in some embodiments, determining the correction factor may be performed using a graph and/or a lookup table that correlates current valve positions with respective correction factors. For example, FIG. 5 is a graph depicting correction factors to be applied to valve adjustments, in accordance with some embodiments of the technology described herein. FIG. 5 illustrates a comparison between a degree to which the valve is currently open (in terms of percentage from 0-100% open) and the applicable correction factor. In the example of FIG. 5, three best-fit curves are illustrated, each curve being for a respective vacuum applied to the gas extraction well. The applicable correction factor may be determined by determining the y-axis position of the curve at the x-axis position corresponding to the current valve position. For example, for a well operating under a vacuum rate of 10 SCFM and a current valve position of 60% open, a correction factor of approximately 10° is selected. By contrast, for a well operating under a maximum flow rate of 10 SCFM and a current valve position of 85% open, a correction factor of approximately 101 is selected. Although in the illustrated embodiment the correction factor is determined using a graph, in other embodiments other techniques may be used. For example, a lookup table may be developed based on the graph shown in FIG. 5. In some embodiments, an equation representing the lines shown in FIG. 5 may be determined and the correction factor may be determined using the equation. Aspects of the techniques described herein are described in terms of using a lookup table, although it should be appreciated that the techniques apply to other methods of determining a correction factor, including use of a graph and/or equation.

In some embodiments, the correction factor may be based on one or more additional factors. For example, as shown in FIG. 5, a maximum flow rate of landfill gas being extracted from the landfill gas extraction well may be a factor in determining the correction factor. That is, a respective one of the three lines may be selected based on the maximum flow rate of landfill gas being extracted from the landfill gas extraction well (e.g., vacuum pressures that would produce flow rates of 10 SCFM, 20 SCFM, 75 SCFM when the valve is fully open) and the correction factor may be determined using the selected line. Accordingly, in some embodiments, the determining the correction factor comprises selecting a lookup table from a plurality of lookup tables based on a maximum flow rate of landfill gas being extracted from the landfill. In some embodiments, a lookup table may be selected from a plurality of lookup tables based on the type of valve implemented in the gas extraction system. That is, the plurality of lookup tables may comprise a respective lookup table for each of a plurality of different valves. In some embodiments, the plurality of lookup tables may correspond to one or more other characteristics, such as an atmospheric characteristic such as temperature, atmospheric pressure, humidity, wind speed, or other characteristic.

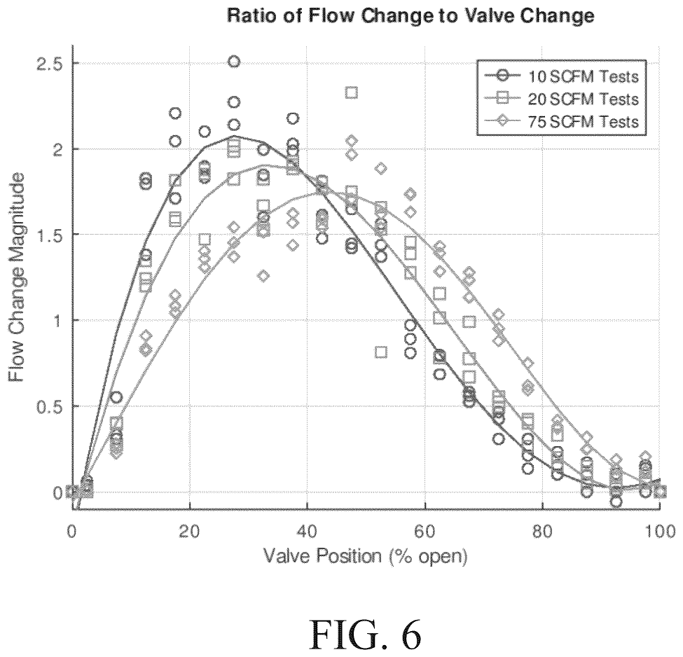

The tools (e.g., graphs, lookup tables, equations) for determining the correction factor may be developed using experimental flow rate data. For example, FIG. 6 is a graph depicting ratios of flow rate changes to valve changes which was used to develop the linearization correction factors graph of FIG. 5. In particular, for each of three maximum flow rates applied to the gas extraction well (10 SCFM, 20 SCFM, and 75 SCFM), a magnitude change in flow rate was recorded for a series of changes in valve positions. The collected data is reflected in the graph of FIG. 6. Best-fit curves for each set of data points were calculated (e.g. using a polynomial fit) to provide the three curves shown in FIG. 6. The resulting curves shown in the graph of FIG. 6 provide an estimate of a resulting magnitude change in flow for respective valve positions along a range of 0-100% open. The linearization correction factor graph of FIG. 5 is developed by taking the reciprocal of the y-axis values and plotting the reciprocals on a logarithmic scale. As described herein, the techniques described herein for determining the valve adjustment can depend on one or more factors. However, the techniques described herein are not limited to use of a lookup table or any other specific data structure for determining the valve adjustment. The valve adjustment may be determined using any suitable technique that is based on one or more factors described herein. Those factors may include the state of the valve which may include characteristics of the valve including the degree to which the valve is open or closed also referred to herein as the current position of the valve, the type of valve, the temperature of the valve, and/or other physical properties of the valve that impact the non-linearity of the valve. Furthermore, one or more additional factors may be used in determining the valve adjustment, as described herein, in addition to the state of the valve. Such features include a vacuum applied to the gas extraction system, one or more characteristics of the landfill gas extracted from the landfill (e.g., a temperature, pressure, gas concentration including methane, carbon dioxide, oxygen, hydrogen sulfide, nitrogen, and/or an estimate of balance gas, an energy content, etc.), one or more characteristics of the gas extraction system (e.g., a pressure in the gas extraction system, a level of liquid in the gas extraction system, a temperature in the gas extraction system), and/or one or more atmospheric conditions (e.g., atmospheric temperature, atmospheric pressure, humidity, wind speed, wind direction, a concentration of greenhouse gas emissions).

At act 408, a corrected valve adjustment is determined using the determined target change in flow rate and the determined correction factor. For example, the corrected valve adjustment may comprise the product of the correction factor and the determined target change in flow rate. The corrected valve adjustment is a valve adjustment that produces a uniform change in flow rate. That is, by implementing the correction factor to change the magnitude of the valve adjustment depending on the current valve position, the resulting change in flow rate can be uniform along the range of valve positions from 0-100% open.

At act 410, the valve is opened or closed by the determined degree according to the corrected valve adjustment.

It should be appreciated that in some embodiments, one or more acts of the process 400 may be omitted. For example, in some embodiments, the method may begin at act 406. It should further be appreciated that in some embodiments the method may only be applied under certain conditions. For example, the process 400 may only be applied if the current position of the valve is within a certain range (e.g., between 5-95%, 10-90%, 15-85%, 20-80%, etc.). Therefore, the process 400 may include an additional act prior to act 410 comprising determining whether the precondition is met before implementing the valve adjustment.

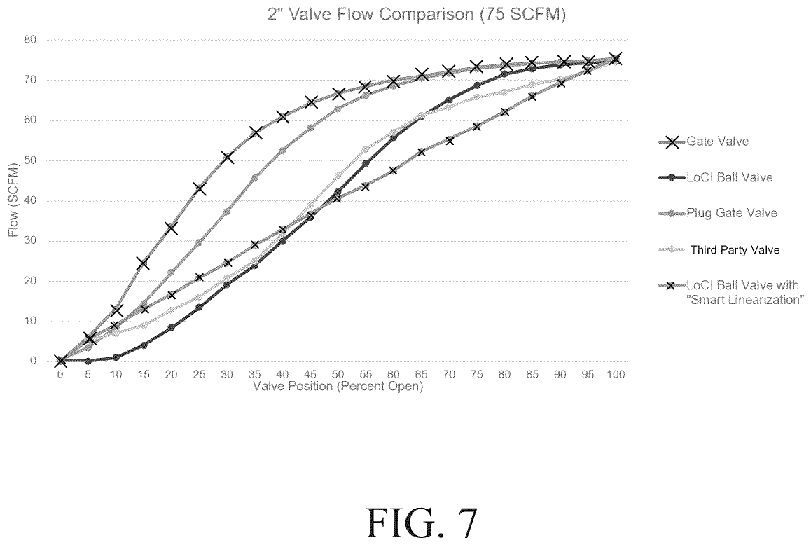

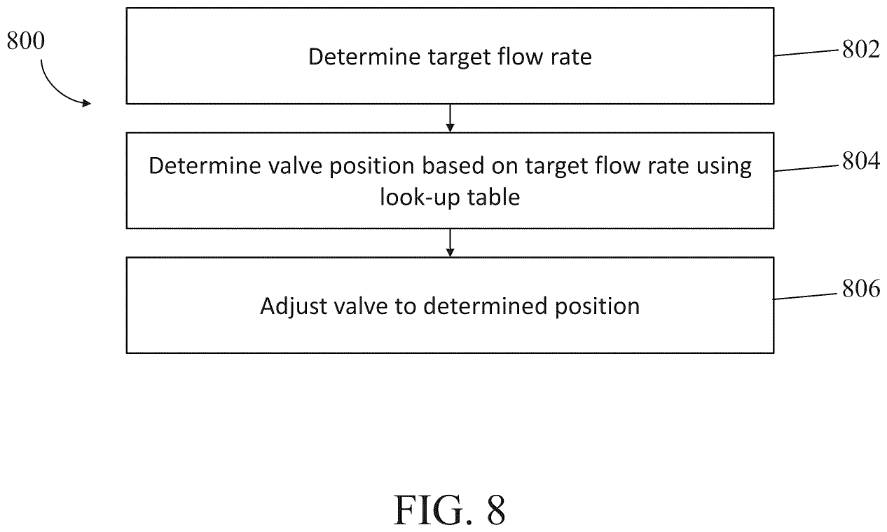

FIG. 7 is a graph comparing linearity of valves, according to some embodiments of the technology described herein. FIG. 7 illustrates the relationship between valve adjustments and changes in flow rate for five valves. The second and fifth curves illustrate results for a ball valve. The fifth curve, however, illustrates results when the techniques for compensating for valve non-linearity described herein are applied. The fifth curve (Ball Valve with “Smart Linearization”) illustrates that application of the techniques described herein provide for improving the linearity of the relationship between valve adjustments and flow rate changes such that changes to flow rate can be uniform across the range of the valve from 0-100% open. In some embodiments, the techniques described herein may be implemented to reach a target flow rate with fewer valve adjustments. For example, the techniques described herein may be used to reach a target flow rate in a single valve adjustment. For example, FIG. 8 illustrates an example process for controlling extraction of landfill gas from a landfill using a technique that compensates for non-linearity in valve adjustments, in accordance with some embodiments of the technology described herein.

The process 800 illustrated in FIG. 8 may begin at act 802 where a target flow rate is determined. For example, the target flow rate may be determined in the same or similar manner as act 404 of process 400 where a target change in flow rate is determined.

At act 804, a valve position is determined based on the target flow rate determined at act 802. The valve position may be determined using a lookup table, in some embodiments. In other embodiments, as described herein, a graph and/or equation representing a curve of the graph may be used in addition or alternative to the lookup table.

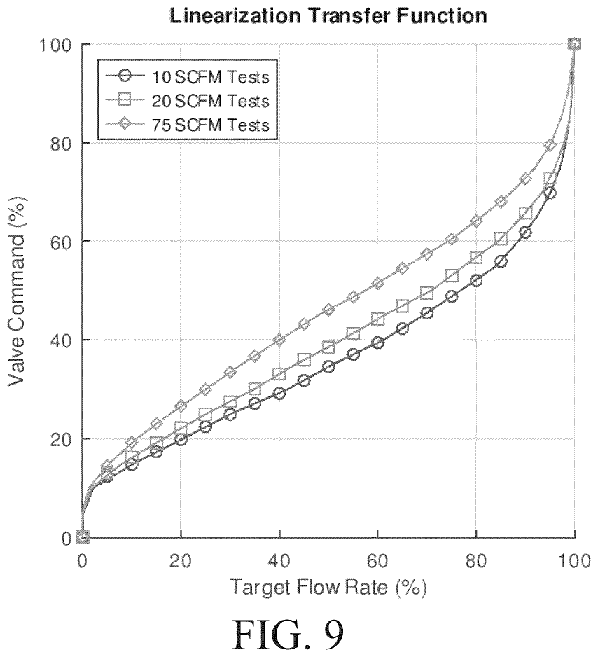

FIG. 9 is a graph depicting a linearization transfer function for use in determining a valve position to achieve a target flow rate, according to some embodiments of the technology described herein. The graph of FIG. 9 may be derived from the data shown in FIG. 6, similar to the graph of FIG. 5 described herein. In particular, FIG. 9 shows the same graph of FIG. 5 with the x-and y-axes being switched.

FIG. 9 is an example of a graph that may be used to determine the valve position. For example, one of the three curves shown in FIG. 9 may be selected based on the vacuum applied to the gas extraction well, which establishes a maximum flow rate. In the example shown in FIG. 9, the three maximum flow rates are 10 SCFM, 20 SCFM, and 75 SCFM. The determined target flow rate obtained at act 802 may then be used to determine a y-axis position along the selected curve that corresponds to the x-axis position corresponding to the determined target flow rate. The y-axis position represents the applicable valve command in terms of percent open. For example, where the maximum flow rate of the gas extraction well is 10 SCFM, the first curve (denoted with circles) is selected. Where the target flow rate is 60%, the valve command is 40%. Accordingly, the target flow rate of 60% can be achieved with a single valve adjustment that positions the valve at 40% open.

At act 806, the valve is adjusted to the determined position. For example, the valve is adjusted (e.g., opened or closed) until the valve is in the position corresponding to the determined valve position.

The techniques described herein may be embodied in software, in some embodiments.

For example, according to some aspects described herein, there is provided at least one non-transitory computer-readable storage medium having instructions encoded thereon that, when executed by at least one processor, cause the at least one processor to execute any one or more of the methods described herein (e.g., process 400, process 800).

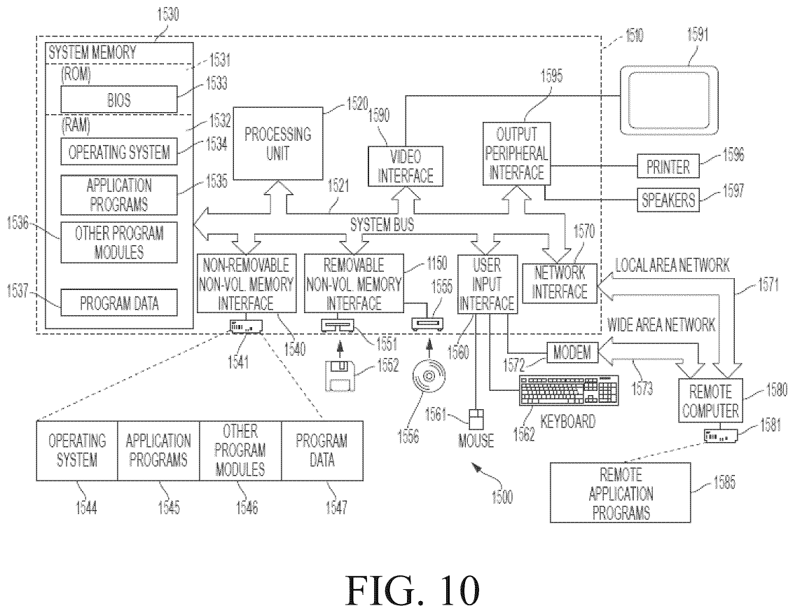

FIG. 10 illustrates an example of a suitable computing system environment 1500 on which techniques disclosed herein may be implemented. In some embodiments, portions of a landfill gas extraction control system may be implemented in a computing system environment. For example, in some embodiments, Device Manager 502, Controller Module 504, User Interface 508, and/or Database 510 may be implemented in a computing system environment. In some embodiments, aspects of one or more techniques described herein may be implemented in a computing system environment.

The computing system environment 1500 is only one example of a suitable computing environment and is not intended to suggest any limitation as to the scope of use or functionality of the devices and techniques disclosed herein. Neither should the computing environment 1500 be interpreted as having any dependency or requirement relating to any one or combination of components illustrated in the exemplary operating environment 1500.

The techniques disclosed herein are operational with numerous other general purpose or special purpose computing system environments or configurations. Examples of well-known computing systems, environments, and/or configurations that may be suitable for use with techniques disclosed herein include, but are not limited to, personal computers, server computers, hand-held devices (e.g., smart phones, tablet computers, or mobile phones), laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The technology described herein may also be practiced in distributed computing environments where tasks are performed by remote processing devices that are linked through a communications network. In a distributed computing environment, program modules may be located in both local and remote computer storage media including memory storage devices.

With reference to FIG. 10, an exemplary system for implementing techniques described herein includes a general purpose computing device in the form of a computer 1510. Components of computer 1510 may include, but are not limited to, a processing unit 1520, a system memory 1530, and a system bus 1521 that couples various system components including the system memory to the processing unit 1520. The system bus 1521 may be any of several types of bus structures including a memory bus or memory controller, a peripheral bus, and/or a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 1510 typically includes a variety of computer readable media. Computer readable media can be any available media that can be accessed by computer 1510 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 1510. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 1530 includes computer storage media in the form of volatile and/or nonvolatile memory such as read only memory (ROM) 1531 and random access memory (RAM) 1532. A basic input/output system 1533 (BIOS), containing the basic routines that help to transfer information between elements within computer 1510, such as during start-up, is typically stored in ROM 1531. RAM 1532 typically contains data and/or program modules that are immediately accessible to and/or presently being operated on by processing unit 1520. By way of example, and not limitation, FIG. 10 illustrates operating system 1534, application programs 1535, other program modules 1536, and program data 1537.

The computer 1510 may also include other removable/non-removable, volatile/nonvolatile computer storage media. By way of example only, FIG. 10 illustrates a hard disk drive 1541 that reads from or writes to non-removable, nonvolatile magnetic media, a magnetic disk drive 1551 that reads from or writes to a removable, nonvolatile magnetic disk 1552, and an optical disk drive 1555 that reads from or writes to a removable, nonvolatile optical disk 1556 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 1541 is typically connected to the system bus 1521 through a non-removable memory interface such as interface 1540, and magnetic disk drive 1551 and optical disk drive 1555 are typically connected to the system bus 1521 by a removable memory interface, such as interface 1550.

The drives and their associated computer storage media described above and illustrated in FIG. 10, provide storage of computer readable instructions, data structures, program modules and other data for the computer 1510. In FIG. 10, for example, hard disk drive 1541 is illustrated as storing operating system 1544, application programs 1545, other program modules 1546, and program data 1547. Note that these components can either be the same as or different from operating system 1534, application programs 1535, other program modules 1536, and program data 1537. Operating system 1544, application programs 1545, other program modules 1546, and program data 1547 are given different numbers here to illustrate that, at a minimum, they are different copies. A user may enter commands and information into the computer 1510 through input devices such as a keyboard 1562 and pointing device 1561, commonly referred to as a mouse, trackball or touch pad. Other input devices (not shown) may include a microphone, joystick, game pad, satellite dish, scanner, or the like. These and other input devices are often connected to the processing unit 1520 through a user input interface 1560 that is coupled to the system bus, but may be connected by other interface and bus structures, such as a parallel port, game port or a universal serial bus (USB). A monitor 1591 or other type of display device is also connected to the system bus 1521 via an interface, such as a video interface 1590. In addition to the monitor, computers may also include other peripheral output devices such as speakers 1597 and printer 1596, which may be connected through an output peripheral interface 1595.

The computer 1510 may operate in a networked environment using logical connections to one or more remote computers, such as a remote computer 1580. The remote computer 1580 may be a personal computer, a server, a router, a network PC, a peer device or other common network node, and typically includes many or all of the elements described above relative to the computer 1510, although only a memory storage device 1581 has been illustrated in FIG. 10. The logical connections depicted in FIG. 10 include a local area network (LAN) 1571 and a wide area network (WAN) 1573, but may also include other networks. Such networking environments are commonplace in offices, enterprise-wide computer networks, intranets and the Internet.

When used in a LAN networking environment, the computer 1510 is connected to the LAN 1571 through a network interface or adapter 1570. When used in a WAN networking environment, the computer 1510 typically includes a modem 1572 or other means for establishing communications over the WAN 1573, such as the Internet. The modem 1572, which may be internal or external, may be connected to the system bus 1521 via the user input interface 1560, or other appropriate mechanism. In a networked environment, program modules depicted relative to the computer 1510, or portions thereof, may be stored in the remote memory storage device. By way of example, and not limitation, FIG. 10 illustrates remote application programs 1585 as residing on memory device 1581. It will be appreciated that the network connections shown are exemplary and other means of establishing a communications link between the computers may be used.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Further, it should be appreciated that various modifications may be made to the specific construction described herein. For example, embodiments are described herein in which a throttle is mechanically configured so as not to fully block a flow path between the well head and gas collection system, even in a position of maximum closure of the throttle. Accordingly, regardless of the output of any control algorithm, so long as the vacuum system is operating as part of the gas collection system, a negative pressure will be maintained at the well head.

It should be appreciated that the same effect may be achieved, for example, by limiting the control signals sent to a control valve. The control signals may be limited so as to preclude control signals that fully close the valve during operation of the gas extraction system.

Further, while a throttle is described as allowing some gas to flow, even in a fully closed position, it may be appreciated that, in some scenarios, it may be desired to fully close off the flow of gas. For example, it may be desired to shut off the gas extraction system for maintenance. To accommodate for such scenarios, one or more valves may be connected to the well piping that may shut off the flow of gas even if the throttle is not fully blocking an aperture through which gas would otherwise flow. In scenarios in which a throttle is implemented by a valve that is controlled so as not to fully close during operation of the gas extraction system, full closure may be achieved, for example, by removing constraints on position of the valve to fully shut of gas flow.

The terms “approximately”, “substantially,” and “about” may be used to mean within ±20% of a target value in some embodiments, within ±10% of a target value in some embodiments, within ±5% of a target value in some embodiments, and within ±2% of a target value in some embodiments. The terms “approximately” and “about” may include the target value.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

What is claimed is:

1. A control system for controlling extraction of landfill gas from a landfill via a gas extraction system, the control system comprising:
a valve configured to control flow rate of landfill gas extracted from the landfill; and
at least one controller configured to:
determine, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and
open or close the valve by the determined degree.

2. The control system of claim 1, wherein the at least one controller is further configured to determine, based on the degree to which the valve is open at the time of the determining, a correction factor, and the determining the adjustment comprises applying the determined correction factor to the target change in flow rate to obtain the adjustment.

3. The control system of claim 1, wherein the at least one controller is further configured to determine the target change in flow rate of landfill gas through the valve.

4. The control system of claim 3, wherein the at least one controller is further configured to:
determine whether to adjust the flow rate of the landfill gas through the valve;
wherein the determining the target change in flow rate through the valve is performed when it is determined to adjust the flow rate of the landfill gas through the valve.

5. The control system of claim 1, wherein the degree to which the valve is open at the time of the determining indicates a position of a throttle of the valve along a range from 0% to 100% open at the time of the determining.

6. The control system of claim 5, wherein the at least one controller is further configured to determine the position of the throttle at the time of the determining.

7. The control system of claim 6, wherein the determining the position of the throttle at the time of the determining is performed based on a record of prior adjustments made to the valve.

8. The control system of claim 2, wherein the determining the correction factor comprises using at least one lookup table that correlates valve positions with respective correction factors.

9. The control system of claim 8, wherein the determining the correction factor further comprises selecting the at least one lookup table from a plurality of lookup tables based on a maximum flow rate of landfill gas being extracted from the landfill and/or a type of the valve.

10. The control system of claim 4, wherein the determining whether to adjust the flow rate of the landfill gas through the valve comprises determining, based on at least one characteristic of the landfill gas, whether to adjust the flow rate of the landfill gas through the valve.

11. The control system of claim 10, wherein the at least one characteristic of the landfill gas comprises a concentration of at least one constituent gas in the landfill gas and/or a pressure in the gas extraction system.

12. The control system of claim 1, wherein the at least one controller comprises:
at least one first controller configured to determine the adjustment to apply to the valve; and
at least one second controller configured to open or close the valve by the determined degree, wherein the at least one second controller is located remotely from the at least one first controller.

13. A method for controlling extraction of landfill gas from a landfill via a gas extraction system comprising a valve configured to control flow rate of landfill gas extracted from the landfill, the method comprising:
determining, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and
opening or closing the valve by the determined degree.

14. The method of claim 13, further comprising:
determining, based on the degree to which the valve is open at the time of the determining, a correction factor,
wherein the determining the adjustment comprises applying the determined correction factor to the target change in flow rate to obtain the adjustment.

15. The method of claim 13, further comprising:
determining the target change in flow rate of landfill gas through the valve.

16. The method of claim 15, further comprising:
determining to adjust the flow rate of the landfill gas through the valve,
wherein the determining the target change in flow rate through the valve is performed when it is determined to adjust the flow rate of the landfill gas through the valve.

17. The method of claim 13, wherein the degree to which the valve is open at the time of the determining comprises a position of a throttle of the valve along a range from 0% to 100% open at the time of the determining.

18. The method of claim 17, further comprising:
determining the position of the throttle at the time of the determining.

19. The method of claim 18, wherein the determining the position of the throttle at the time of the determining is performed based on a record of prior adjustments made to the valve.

20. At least one non-transitory computer-readable storage medium having encoded thereon instructions that, when executed by at least one processor, cause the at least one controller to perform a method for controlling extraction of landfill gas from a landfill via a gas extraction system comprising a valve configured to control flow rate of landfill gas extracted from the landfill, the method comprising:
determining, using a target change in flow rate through the valve and a degree to which the valve is open at the time of the determining, an adjustment to apply to the valve, the adjustment indicative of a determined degree to which to open or close the valve in order to achieve the target change in flow rate of landfill gas through the valve; and
opening or closing the valve by the determined degree.
